(19) United States
(12) Patent Application Publication
(10) Pub. No.: US 2003/0060866 A1
(43) Pub. Date: Mar. 27, 2003

(54) Multi-electrode stimulation parallel to cardiac myofibers

(76) Inventor: John A. Schmidt, Durango, CO (US)

Correspondence Address: GOTTLIEB RACKMAN & REISMAN PC, 270 MADISON AVENUE, 8TH FLOOR, NEW YORK, NY, 100160601

(21) Appl. No.: 09/962,494

(22) Filed: Sep. 25, 2001

(51) Int. Cl. A61N 1/05

(52) U.S. Cl. 607/122

(57) ABSTRACT

A cardiac stimulation system includes multiple electrodes and a cardiac stimulator for generating stimulation pulses. The electrodes are mounted on a tube and stimulation electrodes are designated which are characterized by a optimal stimulation level. Preferably the optimal stimulation level is selected such that the electrical field associated with a stimulation pulse is directed substantially in parallel with the cardiac fibers being stimulated.

### BACKGROUND OF THE INVENTION

A. Field of Invention

This invention pertains to a method and apparatus for applying cardiac stimulation using multiple electrodes, and more particularly, to a method and apparatus which takes advantage of several electrodes in a manner as to minimize the threshold stimulation level.

B. Description of the Prior Art

The heart is a mechanical pump that is stimulated by electrical impulses. The mechanical action of the heart results in the flow of blood. During a normal heartbeat, the right atrium (RA) fills with blood from the returning veins. The RA then contracts and this blood is moved into the right ventricle (RV). When the RV contracts it pumps that blood to the lungs. Blood returning from the lungs moves into the left atrium (LA), and after LA contraction, is pumped into the left ventricle (LV) which then pumps it throughout the body. Four heart valves keep the blood flowing in the proper directions.

The electrical signals that drive this mechanical contraction starts in the sinoatrial node, a collection of specialized heart cells in the right atrium which automatically depolarize (change their voltage potential). This depolarization wave front passes across all the cells of both atria and results in atrial contraction. When the advancing wave front reaches the A-V node it is delayed so that the contracting atria have time to fill the ventricles. The depolarizing wave front then passes over the ventricles causing them to contract and pump blood to the lungs and body. This electrical activity occurs approximately 72 times a minute in a normal individual and is called normal sinus rhythm.

The corresponding electrical signals identifying these events are usually referred to as the P, QRS (or R) and T waves or beats. More particularly, an atrial contraction is represented on an ECG by a P wave, a ventricular contraction is represented by an R wave and a ventricular relaxation is represented by a T wave. The atrium also relaxes but this event is masked by activity in the ventricle and consequently it is not observable on an ECG.

Patients with cardiac deficiency are provided with implantable pacemakers which sense the intrinsic electrical signals of the heart and, if necessary, generate pacing pulses which force a respective cardiac chamber to contract if a contraction is not sensed. On implantation, the pacemaker is programmed with several parameters which defines modes of operation, and other pacing characteristics. One of these parameters is the stimulation threshold level, which is the level of intensity of the pacing pulses required to pace the heart reliably. Two competing interests must be satisfied by selecting this parameter. First, and foremost, the threshold level must be high enough to insure that the heart is indeed captured and paced at the desired rate. However, if the threshold level is too high, the pacing pulses waste energy, always an expensive commodity in an implantable device, and may become uncomfortable for the patient.

Conventional pacemakers utilize a single or dual lead to apply pacing pulses. The dual lead typically includes a tip and a ring electrode. The lead is inserted in such a manner that the tip is imbedded into the cardiac muscle. A pacing pulse is then applied between the tip and the ring electrodes, thereby causing the cardiac muscle to contract. If a single lead is used the electric pulse is applied between the tip electrode and another electrode remotely located, or which may comprise the housing of the pacemaker. As discussed above, these pacing pulses must exceed a threshold which varies from patient to patient.

Recently, articles have been published which indicate that in fact this threshold is not even constant for the same patient, but it varies with the orientation of the induced electric field associated with the electrical pulse with respect to the cardiac muscle fibers (myocytes). More particularly, it was found that threshold levels are highest when the induced electric field is perpendicular to the fibers, and that the threshold is lowest when the induced electric field is parallel to the muscle fibers. (See A L Bardou et al, Directional variability of stimulation threshold measurements in isolated guinea pig cardiomyocytes: Relationship with orthogonal sequential defibrillating pulses (Pace 13:1590, 1990); K B Stokes and G N Kay Artificial Electric Stimulation, (Chapter 1, Clinical Cardiac Pacing, Ellenbogen Ed. W B Saunders 1995). However, existing electrode configurations induce electric fields which have an unknown, and generally random or unpredictable orientation to the muscle fibers. This random orientation is at least one of the sources of the threshold variability.

U.S. Pat. No. 5,824,028 discloses a line electrode oriented parallel to the cardiac fibers. The reference does not mention multi-electrode stimulation. Moreover it appears that the electrode disclosed by this reference applies an electric field which is transversal rather than parallel to the cardiac fibers. Finally, the electrode disclosed by this reference is shaped like a ribbon. This shape is not advisable or practical for the cardiac chamber because it breaks too easily.

### OBJECTIVES AND SUMMARY OF THE INVENTION

In view of the above disadvantages of the prior art, it is an objective of the present invention to provide an implantable cardiac stimulation system, such as a pacemaker, in which two or more electrodes are positioned in a novel arrangement selected to minimize the threshold stimulation level.

A further objective is to provide an implantable cardiac stimulation system with a stimulation level that is optimized to reduce power requirements and power consumption.

Other objectives and advantages of the invention shall become apparent from the following description.

Briefly, the subject invention pertains to an implantable cardiac stimulation system having a cardiac stimulator having electronic circuitry for the stimulation and a multi-electrode lead attached to the stimulator and shaped for insertion into one or more body cavities. (The term cardiac stimulator will be used herein to cover pacemakers as well as other cardiac devices such as internal cardioversion devices and defibrillators). The lead is inserted into the respective cardiac cavities into a predetermined position. Alternatively the lead may be positioned in the veins, or it may be positioned externally of the heart. If the lead has many electrodes, then an appropriate subset of electrodes is selected for stimulation. Advantageously, the set of electrodes include at least two electrodes situated so that they apply stimulation pulses which result in an electric field oriented in parallel to the cardiac muscle tissues. As discussed above, this arrangement is advantageous because it results in a lower stimulation threshold level, and hence in a lower power requirement and extended battery life. In one embodiment, the multiple electrodes comprise axially spaced rings arranged along a common tubular rod. In another embodiment, the electrodes comprised radially spaced dots disposed along the tubular rod. In a third embodiment, the lead comprises an enlarged, mushroom shaped head composed of slotted electrodes being disposed on the head and angularly displaced and electrically isolated from one another.

More specifically, an implantable cardiac stimulation system is disclosed with a stimulator adapted to sense intrinsic cardiac activity and to generate a stimulation pulse responsive to intrinsic cardiac activity, said stimulation pulse having an amplitude associated with a stimulation threshold; and a plurality of implanted electrodes including a pair of electrodes selected based on their characteristic stimulation threshold. The electrodes are implanted in a patient's heart having cardiac fibers defining a fiber direction, wherein the pair of electrodes generates an electric field in the presence of said stimulation pulse, said electrical field extending substantially parallel to said fiber direction.

The electrodes can be partitioned into several electrode families, including a stimulation family selected for applying said stimulation pulse, wherein the electrode pair is selected from said stimulation family.

In a preferred embodiment, a lead having an elongated member is provided with the electrodes being formed on said elongated member. The electrodes comprise axially spaced rings disposed on said elongated member, each ring being connected to a wire extending though said elongated member. The elongated member may be a tube housing the wires. The electrodes can be angularly spaced with respect to each about the elongated member. The tube may include an elongated cavity adapted to receive a removable stylet. The stylet may be more rigid then the lead and may be used for the implantation of the lead. After the lead is implanted, the stylet is removed.

In another aspect of the invention, a cardiac stimulator having an elongated lead having a distal and a proximal end, said distal end being adapted to be implanted in the heart of a patient, said lead including a plurality of electrodes including a pair of stimulation electrodes; and a cardiac stimulator connected to said proximal end and including a stimulation pulse generator to generate a stimulation pulse, said stimulator being adapted to apply said stimulation pulse to said stimulation electrode pair, said stimulation pulse having a characteristic dependent on a stimulation threshold. The electrode pair is selected to optimize stimulation threshold.

In another aspect of the invention, a method is presented for designating stimulating electrodes from a plurality of electrodes in a cardiac stimulation system, the method including implanting the plurality of electrodes; determining for subsets of said electrodes corresponding stimulating threshold levels; selecting an optimal threshold level from said stimulating threshold levels; and designating the subset of electrodes corresponding to said optimal threshold as electrodes for applying cardiac stimulation. Preferably the optimal threshold level is the smallest threshold level. The threshold levels are determined by applying sequentially stimulation pulses to the respective electrode sets and sensing the resulting cardiac stimulation. Moreover, the method may further include reducing a characteristic of said sequential pulses until no resulting stimulation is sensed. The method further includes determining the physical location of the electrodes and the location of said electrodes with respect to internal cardiac walls of the heart in which said plurality of electrodes is implanted.

Preferably, the physical location is determined by applying a test signal having a frequency selected from signals that have no effect on cardiac tissues and sensing a delay in and/or amplitude of the signals resulting in the electrodes from said test signal.

In another aspect of the invention, a method of designating stimulating electrodes from a plurality of electrodes in a cardiac stimulator is presented consisting of implanting the plurality of electrodes; determining for a subset of said electrodes which, when receiving a stimulation pulse, generates an electric stimulation field having a field direction in relation to the fiber direction of the cardiac tissues to be stimulated, said subset of electrodes being designated to be the stimulation electrodes to deliver stimulation signals to the cardiac tissues. Preferably wherein said subset of electrodes is designated so that it applies an electrical field substantially parallel to the fiber direction. The method further includes generating a stimulation pulse, and analyzing the resulting signals from the electrodes. Preferably during this process a characteristic (such as its amplitude) is changed until no resulting signals are sensed from the stimulation signal.

### BRIEF DESCRIPTION OF THE INVENTION

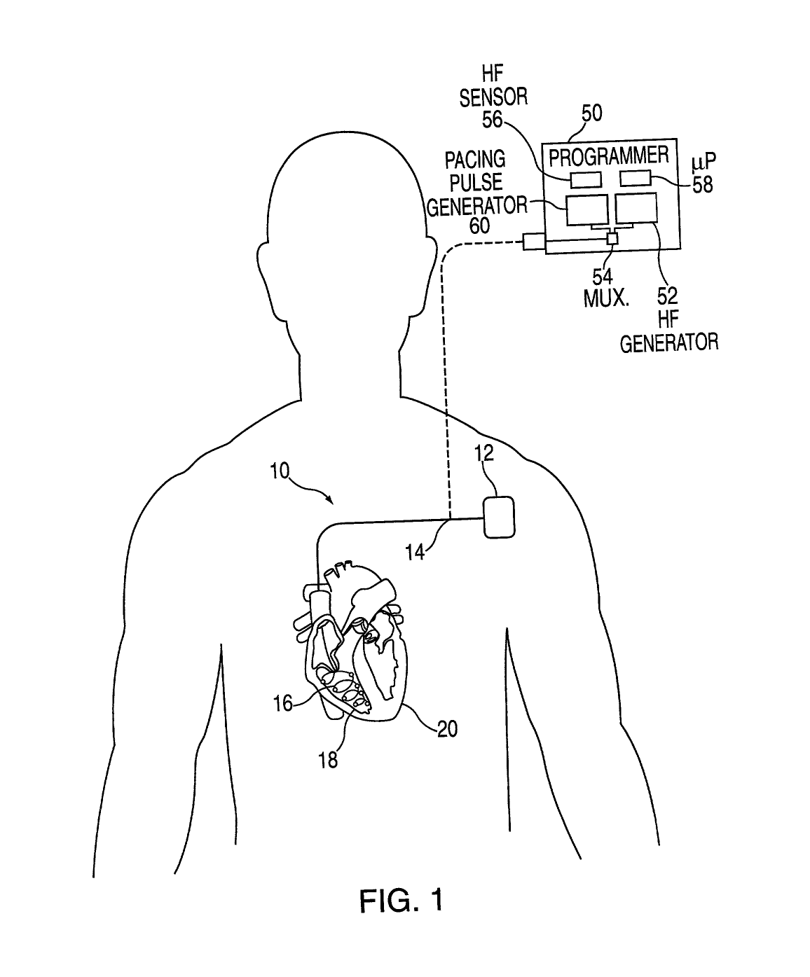

FIG. 1 shows a somewhat diagrammatic front view of a patient with a cardiac stimulation system in accordance with this invention, and including a programmer used to program the cardiac stimulator after the electrodes are implanted.

FIG. 2 shows a side elevational view of a multi-electrode lead used in the cardiac stimulation system of FIG. 1;

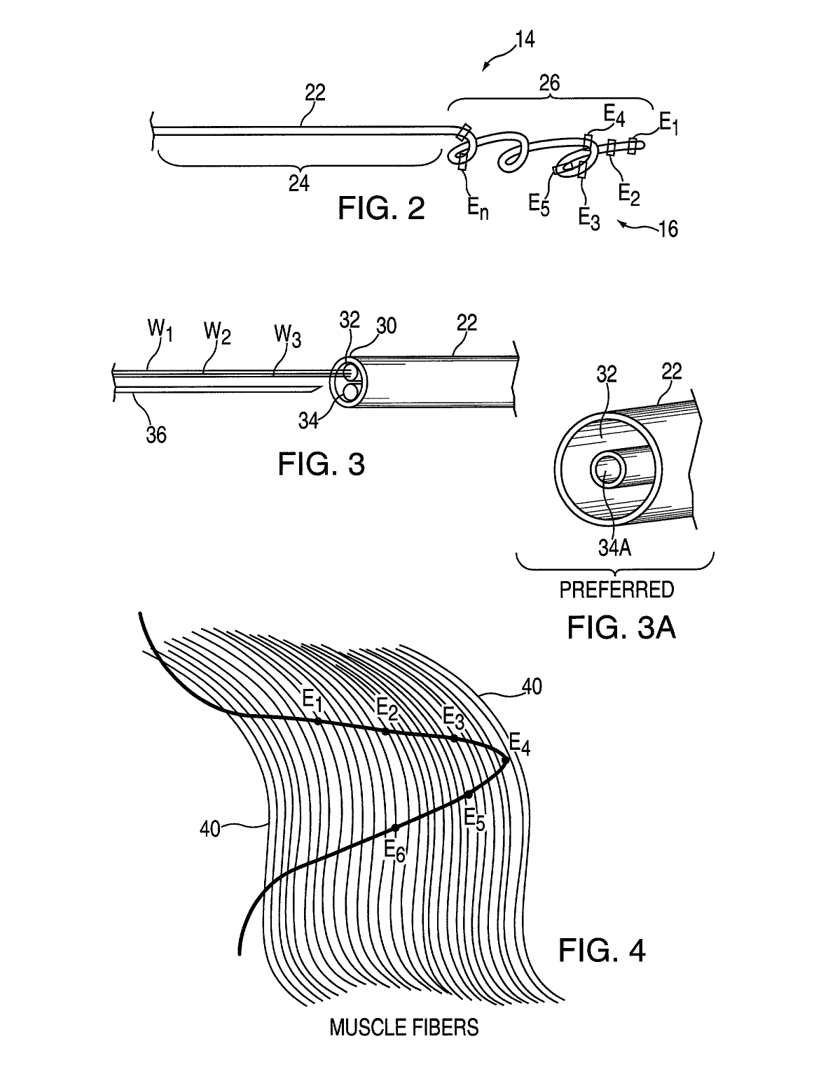

FIG. 3 shows a side elevational view of the proximal end of the lead of FIG. 2;

FIG. 3A shows an alternate embodiment of the proximal end of the lead;

FIG. 4 shows a somewhat diagrammatic view of a portion of the lead of FIGS. 1-3 and the orientation of electrodes thereon with respect to cardiac fibers;

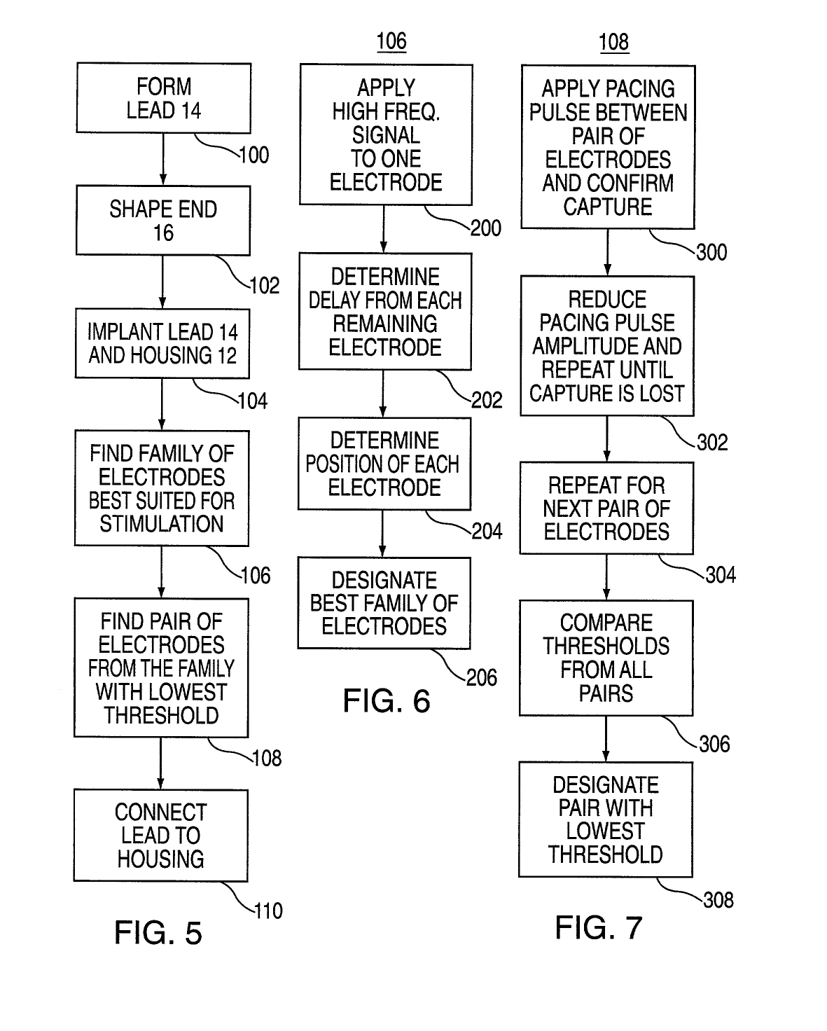

FIG. 5 shows a general flow chart for designating stimulation electrodes in a multi-electrode lead;

FIG. 6 shows a detailed flow chart for determining the positions of the electrode in a cardiac cavity, and for designating sets or families of electrodes having specific locations;

FIG. 7 shows a detailed flow chart for designating stimulation electrodes within a predetermined set or family of electrodes;

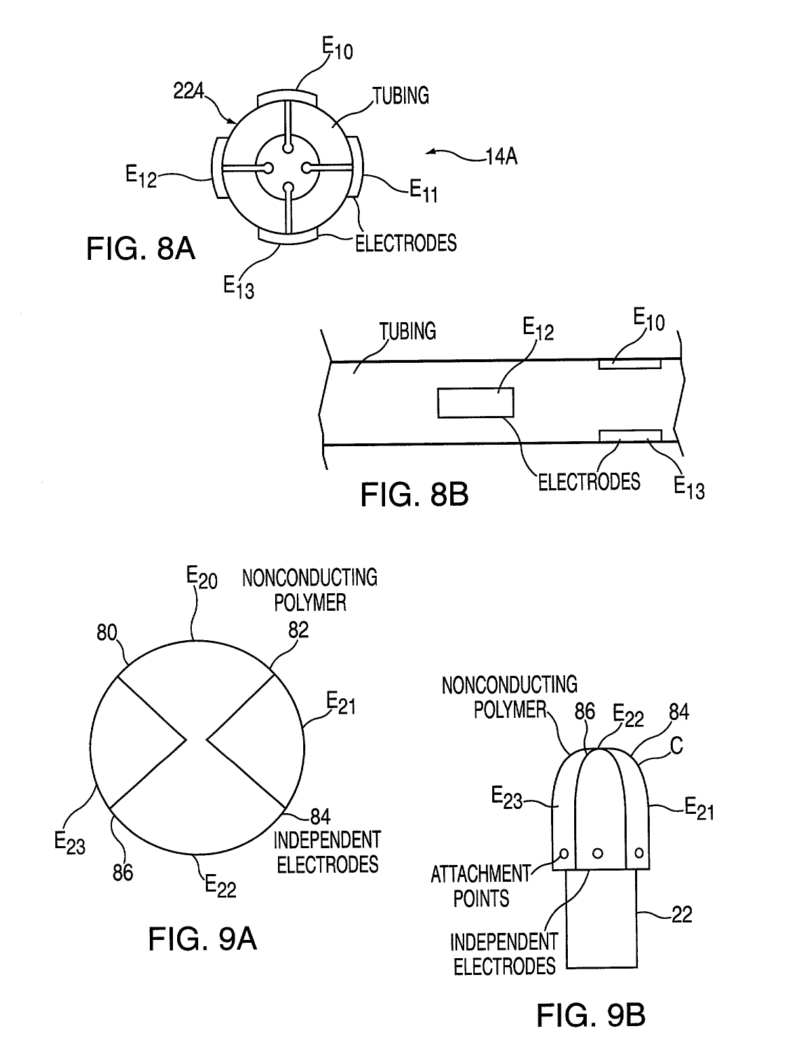

FIG. 8A shows a cross sectional view of an alternate embodiment of the multi-electrode lead;

FIG. 8B shows a partial side elevational view of the embodiment of FIG. 8A;

FIG. 9A shows a cross sectional view of another embodiment of the multi-electrode lead;

FIG. 9B shows a partial side elevational view of the embodiment of FIG. 9A; and

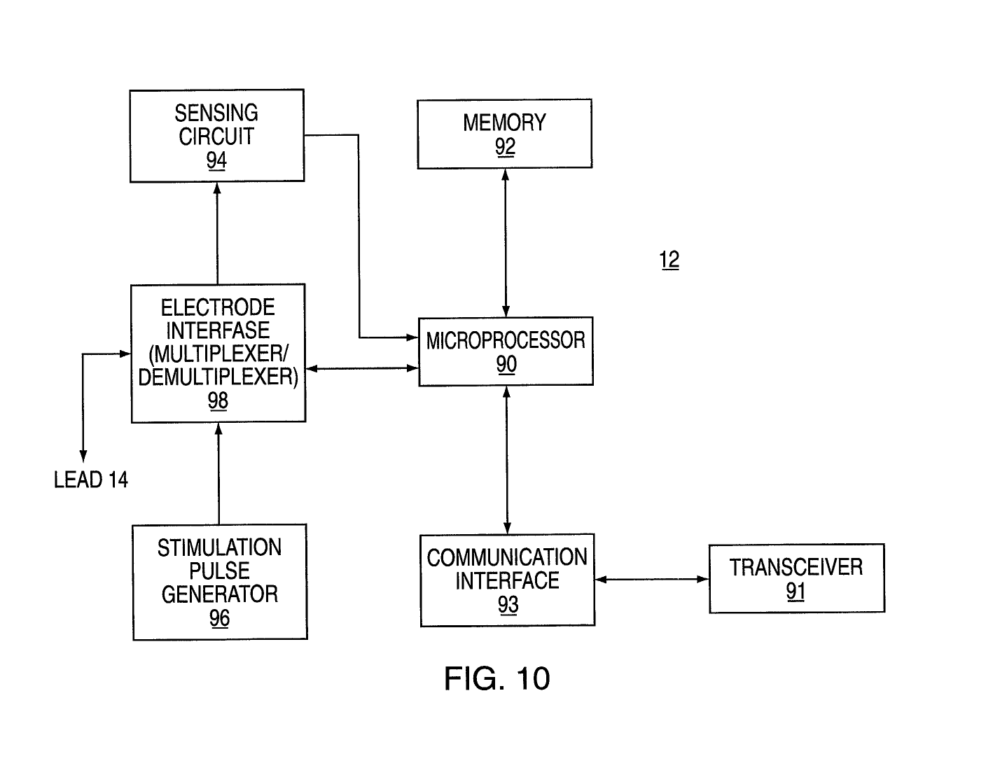

FIG. 10 shows 10 shows a block diagram of the device used in the cardiac stimulator of FIG. 1.

### DETAILED DESCRIPTION OF THE INVENTION

The subject invention pertains to an implantable cardiac stimulation system 10 including a cardiac stimulator 12 with various electronic circuits, and a multi-electrode lead 14 attached to the generator 12, as shown. The lead 14 has a distal end 16 disposed, for example, in one of the cardiac chambers such as the right ventricle 18 of heart 20. In FIG. 1, end 16 is shown having a general spiral shape. The system 10 is adapted to deliver therapy in the form of electrical pulses. The therapy may include GCV (greater cardiac vein) resynchronization therapy, treatment of conduction pathway abnormalities, bardycardia pacing, etc.

Details of the multi-electrode lead 14 are shown in FIG. 2. In a somewhat preferred embodiment, the lead 14 includes an external biocompatible polymer tube 22 having a straight portion 24 and a shaped portion 26. The tube may be made of polyurethane or other similar materials which may be thermally shaped so that its portion 26 retains any desired configuration. In FIGS. 1 and 2 the portion 26 is shown as having a spiral shape, but many other shapes may be selected as well. Attached to tube 22, preferably on portion 26 there are provided a plurality of electrodes E1, E2, E3, E4, E5, . . . En. Preferably electrodes E1 . . . En are formed of coils of bare wire or cable wound about the tube 22. Each electrode is connected to a corresponding wire W1, W2, W3 etc. which extend through the length of tube 22 and are shown as exiting through end 30 for the sake of clarity. Wires W1, W2, W3 . . . are insulated so that they are not shorted to each other within the tube 22. The electrode 14 and its method of manufacture are disclosed in copending commently assigned application Ser. No. 09/245,246 filed Feb. 5, 1999, and incorporated herein by reference. Preferably the end 30 of tube 22 and the ends of wires W1, W2, W3, etc. are coupled to a connector (not shown) for attaching the lead 14 to the cardiac stimulator 12. In this manner the lead 14, can be constructed with the tube 22 extending relatively straight and then can be customized to any shape to fit any preselected location within the heart 20 dependent on each particular patient's pathology. For example, if the lead 14 is to be placed in the greater cardiac vein, then its end 16 (consisting of tube portion 26 and electrodes E1, E2, E3 . . . etc.) is shaped to form a small helix, as shown so that it will fit into said vein.

The tube 22 can be formed with two substantially co-extensive longitudinal cavities 32, 34. Cavity 32 is used to hold the wires W1, W2, W3 etc. Cavity 34 is used to straighten the end 16 before the lead 14 is implanted. For example, the lead 14 could be straightened by inserting a substantially straight stylet 36 into cavity 34. The stylet 36 is also flexible but is less flexible then the lead 14 so that as it is inserted into the cavity 34, it forces the tube 22 to straighten. The lead 14 is now threaded into the heart or vein associated therewith together with the stylet 36. After implantation of the lead 14, the stylet 36 is withdrawn and the lead 14 flexes back and takes the configuration shown in FIG. 2.

In a somewhat preferred embodiment the tube 22 may have a single central cavity 32 for the wires and the stylet 36, with the wires being disposed in an open tube 32 and the stylet being inserted into sheath 34A as shown in FIG. 3A.

FIG. 4 shows a section of end 16 with electrodes E1,E2 . . . E6 being disposed near a set of cardiac tissue fibers 40. If a bipolar stimulation pulse is applied between any two of these electrodes, the orientation of the resulting electric field with respect to the fibers 40 varies from pair to pair. For example, a stimulation pulse between electrodes E1 and E2, or E5 and E6 generates an electric field which is at a considerable angle, i.e., close to 90°. On the other hand, pulses between some of the other electrodes, for example, between E2 and E6 or E3 and E5 result in respective electric fields that are substantially parallel to the fibers 40. Thus, according to the invention, in order to insure an optimized or minimum stimulation threshold, the electrodes for stimulation are selected which align the resulting electric field most efficiently.

The process for implanting system 10 is now described in conjunction with FIGS. 5, 6 and 7. Starting in FIG. 5, first the lead 14 is formed in step 100. In step 102 the lead is shaped, for example to the configuration shown in FIG. 3. In step 104 the shaped lead 14 and cardiac stimulator 12 are implanted. If a stylet is used to implant the lead 14, then as part of step 104 the styled is removed, a connector (not shown) is attached to the end 30 of the tube 22 and the lead 14 is then connected to an external programmer or similar device (not shown) so that the electrodes can be properly located, identified and designated for specific purposes. More particularly, some the electrodes may be designated as sensing electrodes, while other electrodes may be designated as pacing electrodes. However, in the present application, only the designation of the pacing electrodes is of interest.

In step 108 a family of electrodes is designated as being best suited for stimulation based on a set of preselected criteria and tests that may be performed on the electrodes. For example, the designated family may be the set of electrodes E1-E6 of FIG. 4.

Next, in step 108, the family of electrodes are analyzed in pairs, and the pair with the lowest stimulation threshold is designated as the stimulation electrodes.

In step 110 the lead 14 is disconnected from the programmer and connected to the stimulator 12 and the system is then initialized for operation.

The process of designating the family of stimulating electrodes may be performed using many different approaches. Preferably, the steps of designating the electrodes includes a phase during which the location of the electrodes on the lead are determined. As described above, after implantation, the free end of lead 14 is connected to programmer 50, as shown in dotted in FIG. 1. Next, in step 200(See FIG. 6) a high frequency test signal is fed to one of the electrodes, such as the electrode disposed at the tip of the lead 14. Preferably this test signal has a frequency in a range which is known to have no effect on the heart 20. For example, the test signal may have a frequency of about 200 MHz. This test signal is generated by a high frequency or HF generator 52 and applied to the lead 14 by a multiplexer 54. While this HF test signal is applied to the one lead electrode, a sensor 56 within the programmer 50 is used to detect the HF signals in the remaining electrodes. In step 202, a microprocessor 58 is used to determine the delay between the detected signals in each electrode and the HF test signal. (Step 202). Using this information, the microprocessor 58 then determines the position of each electrodes in step 204 with respect to the interior walls of the respective cardiac chamber. Details of the algorithms used to make this determination are provided in commonly assigned copending application Ser. No. 60/288,358 filed May 3, 2001 and entitled “Implantable Electrode System To Map Electrical Activity In 3-Dimensions And Deliver Multifocal Pacing Therapy For Atrial Fibrillation”, incorporated herein by reference. Briefly, as described in that application, a 3D electrode positioning system operates by applying a periodic voltage to several subgroups of the electrodes and measuring the signal induced on selected remaining electrodes. The number of subgroups employed is sufficient to overdetermine a multipole electrical distribution model of the electrodes and surrounding tissue. Several means are available to extract the electrode positions relative to tissue boundaries from such models. One of these, the method of non-linear least squares, is well-known in the literature. (e.g. Golub and Van Loan, Matrix Computations, 1989 Johns Hopkins)

In step 206, families of sensing and pacing electrodes are designated. This may be done automatically, using predetermined rules provided in programmer 50. Alternatively, the electrode families may be designated by a physician based on the position of the electrodes and other criteria. Using this process, it may be determined for example, that the electrodes E1-E6 of FIG. 4 should be used for ventricular pacing.

Details of step 108 are provided in FIG. 7. The purpose of this process is to determine which electrodes of a particular family, such as the family of electrodes E1-E6 of FIG. 4. For this purpose, the lead is still connected to programmer 50 and in step 300 a pacing pulse is applied between a pair of electrodes of the same family, for example E1 and E2. This pacing pulse is generated by a pacing pulse generator 60 and applied between electrode pairs by multiplexer 54. The amplitude or energy content of the pacing pulse is selected to insure that the heart is captured. The sensor 66 is used to confirm that the applied pacing pulse has captured the respective cardiac chamber (in this case, the ventricle). Next, in step 302, the amplitude of the pacing pulse is reduced, the pacing pulse is applied to the same electrode pair and capture is confirmed. Step 302 is repeated until capture is lost. This is a standard method of determining the threshold pacing level for electrodes E1-E2. In step 304 the process is repeated for all the other pairs of the family, for example, E1-E3, E1-E4, E2-E3, E2-E4, . . . etc.

In step 306 the threshold levels thus obtained for all the electrode pairs are compared by the microprocessor 58. Based on these comparisons, in step 308, the pair with the lowest threshold (for example, E2-E6) is designated as the pair to be used for pacing. It is expected that, based on the selection of the electrode family, and the proximity of the electrodes, the chosen electrode pair will generate an electric field parallel to the muscle fibers 40 as discussed above.

The invention has been described so far in conjunction with a multiple electrode lead comprising axially spaced electrodes formed of rings extending around the tube 22 (FIG. 2). However, multi-electrode leads may have other configurations and the present application is equally applicable to these other types of leads as well. More particularly, as shown in FIGS. 8A and 8B, a lead 14A may be provided which includes a hollow tube 22A having a substantially circular cross section, with a plurality of electrodes E10, E11, E12 and E13 extend only partially around the tube 22A being angularly offset from each other. The electrodes are spot welded to the end of respective wires W. The electrodes E10-E13 could be offset 90° and disposed in a plane transversal to the longitudinal axis of tube 22A. Alternatively, as can be seen in FIG. 8B, two of the electrodes could be disposed at 180° while the other two electrodes E11 and E12 (only E12 being visible in FIG. 8B) being offset angularly with respect to each other and the pair E10-E13 being axially spaced from pair E11-E12. Using the process of FIGS. 5-7, the electrodes E10-E13 can be designated as an electrode family and a pair of this family can then be designated or selected as the pacing pair. Leads 22 and 22A can be made with up to 64, 128 or even 256 electrodes.

Another embodiment of the invention is show in FIGS. 9A and 9B. In this Fig., lead 14B includes a tube 22B terminating in a cap C. Cap C is partitioned into a plurality of electrodes E20-E23. Each of these electrodes is welded to a respective wire (not shown) extending through the tube 22B. The electrodes are separated by insulators 80, 82, 84, 86. These electrodes can constitute an electrode family which then may be analyzed to designated for pacing using the techniques discussed above.

The selection of optimal electrodes has been described in conjunction with pacing of the right ventricle. However, the same techniques may be used for other types of stimulations as well, including atrial pacing, dual chamber pacing, atrial and ventricular cardioversion, atrial defibrillation, etc. Moreover, while the techniques are described in conjunction with a single multi-electrode lead, they are applicable for leads having other configurations, such as several single or multi-electrode leads.

FIG. 10 shows a block diagram of the elements disposed in cardiac stimulator 12. The cardiac stimulator 12 includes a microprocessor 90 which operates in accordance with a program stored into memory 92. The cardiac stimulator further includes a sensing circuit 94, a stimulation pulse generator 96 and an electrode interface 98. The electrode interface 98 is connected to the electrodes through lead 14. The electrode interface 98 may be implemented as a multiplexer/demultiplexer combination. The programming in memory 92 is received through a transceiver 91 (for instance from programmer 50) and communication interface 93. As part of this programming, the electrodes designated for stimulation, as described above, are stored in memory 92.

During its operation, the microprocessor 90 sets the electrode interface 98 in accordance with the appropriate electrode designations. Thereafter, the sensing circuit 94 senses intrinsic activity and other signals within the heart 20 and provides corresponding indication signals to the microprocessor 90. The microprocessor 90 then issues appropriate commands to the pulse generator 96. The pulse generator 96 generates appropriate stimulation pulses. These pulses are steered to the designated electrode pair by the electrode interface 98.

Numerous other modifications may be made to this invention without departing from its scope as defined in the attached claims.

We claim:

1. An implantable cardiac stimulation system comprising:
a cardiac stimulator adapted to sense intrinsic cardiac activity and to generate a stimulation pulse responsive to intrinsic cardiac activity, said stimulation pulse having an amplitude associated with a stimulation threshold; and
a plurality of implanted electrodes including a pair of electrodes selected to optimize said stimulation threshold.

2. The cardiac stimulation system of claim 1 wherein said electrodes are implanted in a patient's heart having cardiac fibers defining a fiber direction, wherein said pair of electrodes generates an electric field in the presence of said stimulation pulse, said electrical field extending substantially parallel to said fiber direction.

3. The cardiac stimulation system of claim 1 wherein said plurality of electrodes is partitioned into several electrode families, including a stimulation family selected for applying said stimulation pulse, wherein said electrode pair is selected from said stimulation family.

4. The cardiac stimulation system of claim 1 further comprising a lead having an elongated member with said electrodes being formed on said elongated member.

5. The cardiac stimulation system of claim 4 wherein said electrodes comprise axially spaced rings disposed on said elongated member, each ring being connected to a wire extending though said elongated member.

6. The cardiac stimulation system of claim 4 wherein said elongated member is a tube.

7. The cardiac stimulation system of claim 4 wherein said electrodes are angularly spaced with respect to each about said elongated member.

8. The cardiac stimulation system of claim 7 wherein a set of said electrodes are disposed in a single plane transversal to a longitudinal axis of said elongated member.

9. The cardiac stimulation system of claim 7 wherein said electrodes include a first and a second set of electrodes, the electrodes of each set being angularly spaced, the first set being axially spaced from the second set.

10. The cardiac stimulation system of claim 7 wherein said lead includes a cap formed with said plurality of electrodes.

11. The cardiac stimulation system of claim 10 wherein said electrodes are radially spaced with respect to each other.

12. A cardiac stimulation system comprising:
an elongated lead having a distal and a proximal end, said distal end being adapted to be implanted in the heart of a patient, said lead including a plurality of electrodes including a pair of stimulation electrodes; and
a cardiac stimulator connected to said proximal end and including a stimulation pulse generator to generate a stimulation pulse, said cardiac stimulator being adapted to apply said stimulation pulse to said stimulation electrode pair, said stimulation pulse having a characteristic dependent on a stimulation threshold;
wherein said electrode pair being selected to optimize said stimulation threshold.

13. The stimulation system of claim 12 wherein said elongated lead includes a tube and said electrodes comprise rings axially spaced on said tube and a plurality of wires extending through said tube, each wire being connected to one of said rings.

14. The stimulation system of claim 12 wherein said elongated lead includes a tube having a longitudinal axis and said electrodes are mounted on said lead in electrode groups, each electrode group including electrodes angularly spaced about said tube.

15. The stimulation system of claim 14 wherein said electrode groups are axially separated from each other.

16. The stimulation system of claim 12 wherein said tube is provided with an end portion, said electrodes being mounted on said end portion.

17. The stimulation system of claim 16 wherein said end portion includes a curved cap, said electrodes being supported on said curved cap.

18. A method of designating stimulating electrodes from a plurality of electrodes in a cardiac stimulator comprising:
implanting the plurality of electrodes;
determining for subsets of said electrodes corresponding stimulating threshold levels;
selecting an optimal threshold level from said stimulating threshold levels; and
designating the subset of electrodes corresponding to said optimal threshold as electrodes for applying cardiac stimulation.

19. The method of claim 18 wherein said optimal threshold level is the smallest threshold level.

20. The method of claim 18 wherein said threshold levels are determined by applying sequentially stimulation pulses to the respective electrode sets and sensing the resulting cardiac stimulation.

21. The method of claim 20 further comprising reducing a characteristic of said sequential pulses until no resulting stimulation is sensed.

22. The method of claim 18 further defining from said plurality of electrodes a family of electrodes suitable for applying stimulation, said subsets being selected from said family.

23. The method of claim 22 further comprising determining a physical location for each electrode based on said resulting signals.

24. The method of claim 23 wherein said physical location is determined from a delay between said test signal and said resulting signals.

25. The method of claim 18 further comprising determining the physical location of the electrodes.

26. The method of claim 25 further comprising determining the location of said electrodes with respect to internal cardiac walls of the heart in which said plurality of electrodes is implanted.

27. The method of claim 18 further comprising applying a test signal having a frequency selected from signals that have no effect on cardiac tissues.

28. The method of claim 27 further comprising applying a test signal in the range of 200 MHz.

29. The method of claim 28 further comprising sensing a delay in the signals resulting in the electrodes from said test signal.

30. The method of claim 29 further comprising determining a physical location of said electrodes from the resulting signals.

31. The method of claim 30 further comprising designating a family of stimulating electrodes from said plurality of electrodes based on said physical location.

32. A method of designating stimulating electrodes from a plurality of electrodes in a cardiac stimulator comprising:
implanting the plurality of electrodes;
determining for a subset of said electrodes which, when receiving a stimulation pulse, generates an electric stimulation field having a field direction in relation to the fiber direction of the cardiac tissues to be stimulated, said subset of electrodes being designated to be the stimulation electrodes to deliver stimulation signals to the cardiac tissues.

33. The method of claim 32 wherein said subset of electrodes is designated so that it applies an electrical field substantially parallel to the fiber direction.

34. The method of claim 32 wherein said subset of electrodes is designated by generating a stimulation pulse, and analyzing the resulting signals from the electrodes.

35. The method of claim 34 further comprising generating said stimulation pulse sequentially and changing a characteristic of said stimulation pulse.

36. The method of claim 35 further comprising changing said parameter until no resulting signals are sensed.
